SEQUENCE LISTING (151 sequences)

SEQ ID NO: 1 - DNA, 20 bp, synthetic construct
  source 1..20: mol_type = other DNA
ccaaacctagcctattatcc

SEQ ID NO: 2 - DNA, 20 bp, synthetic construct
  source 1..20: mol_type = other DNA
agctctaataaaccgagcac

SEQ ID NO: 3 - DNA, 20 bp, synthetic construct
  source 1..20: mol_type = other DNA
ccctcctagcccgacgtgac

SEQ ID NO: 4 - DNA, 20 bp, synthetic construct
  source 1..20: mol_type = other DNA
ggccctaggtgaggatgtca

SEQ ID NO: 5 - DNA, 20 bp, synthetic construct
  source 1..20: mol_type = other DNA
ccatctaagatagcagcagc

SEQ ID NO: 6 - DNA, 20 bp, synthetic construct
  source 1..20: mol_type = other DNA
cctagctacttgggagtctg

SEQ ID NO: 7 - DNA, 20 bp, synthetic construct
  source 1..20: mol_type = other DNA
tctctagagatggtttatca

SEQ ID NO: 8 - DNA, 20 bp, synthetic construct
  source 1..20: mol_type = other DNA
agaatctctatgtcttttgg

SEQ ID NO: 9 - DNA, 20 bp, synthetic construct
  source 1..20: mol_type = other DNA
tttggctacttggtctcttc

SEQ ID NO: 10 - DNA, 20 bp, synthetic construct
  source 1..20: mol_type = other DNA
gatgcttctagaagcctgga

SEQ ID NO: 11 - DNA, 20 bp, synthetic construct
  source 1..20: mol_type = other DNA
ttcgtccctagctctgaagg

SEQ ID NO: 12 - DNA, 20 bp, synthetic construct
  source 1..20: mol_type = other DNA
cctttcctaagacattgcta

SEQ ID NO: 13 - DNA, 20 bp, synthetic construct
  source 1..20: mol_type = other DNA
tggaggctactccagaacca

SEQ ID NO: 14 - DNA, 20 bp, synthetic construct
  source 1..20: mol_type = other DNA
gaaaagctatactctagtgg

SEQ ID NO: 15 - DNA, 20 bp, synthetic construct
  source 1..20: mol_type = other DNA
tgtcctacagctttgtccag

SEQ ID NO: 16 - DNA, 20 bp, synthetic construct
  source 1..20: mol_type = other DNA
ttcagttctaagtcaacgtt

SEQ ID NO: 17 - DNA, 20 bp, synthetic construct
  source 1..20: mol_type = other DNA
atctcctaattcttcacaaa

SEQ ID NO: 18 - DNA, 20 bp, synthetic construct
  source 1..20: mol_type = other DNA
tgcctactcattttcttcac

SEQ ID NO: 19 - DNA, 20 bp, synthetic construct
  source 1..20: mol_type = other DNA
atggtggctatccacgatgg

SEQ ID NO: 20 - DNA, 20 bp, synthetic construct
  source 1..20: mol_type = other DNA
atagctaaacactcatcatt

SEQ ID NO: 21 - DNA, 20 bp, synthetic construct
  source 1..20: mol_type = other DNA
gccaactatggcgtgacaga

SEQ ID NO: 22 - DNA, 20 bp, synthetic construct
  source 1..20: mol_type = other DNA
tcattctatttgcctttttt

SEQ ID NO: 23 - DNA, 20 bp, synthetic construct
  source 1..20: mol_type = other DNA
gctttctagcagcctcccca

SEQ ID NO: 24 - DNA, 20 bp, synthetic construct
  source 1..20: mol_type = other DNA
ttgtgctacagactaaatcc

SEQ ID NO: 25 - DNA, 20 bp, synthetic construct
  source 1..20: mol_type = other DNA
acaactaaagttctgagcta

SEQ ID NO: 26 - DNA, 20 bp, synthetic construct
  source 1..20: mol_type = other DNA
gattcctaagactgtttgct

SEQ ID NO: 27 - DNA, 20 bp, synthetic construct
  source 1..20: mol_type = other DNA
cttttcctagtgcccatacc

SEQ ID NO: 28 - DNA, 20 bp, synthetic construct
  source 1..20: mol_type = other DNA
caaggctaagtcccacgtgc

SEQ ID NO: 29 - DNA, 20 bp, synthetic construct
  source 1..20: mol_type = other DNA
caatcttctatttctctaaa

SEQ ID NO: 30 - DNA, 20 bp, synthetic construct
  source 1..20: mol_type = other DNA
tcctcgctacaggaagctgc

SEQ ID NO: 31 - DNA, 20 bp, synthetic construct
  source 1..20: mol_type = other DNA
tctttcctatttcttcacac

SEQ ID NO: 32 - DNA, 20 bp, synthetic construct
  source 1..20: mol_type = other DNA
gaaacagctacaaaaccagt

SEQ ID NO: 33 - DNA, 20 bp, synthetic construct
  source 1..20: mol_type = other DNA
gagctctacgtcagcttcca

SEQ ID NO: 34 - DNA, 20 bp, synthetic construct
  source 1..20: mol_type = other DNA
gggctagttgaatttagcct

SEQ ID NO: 35 - DNA, 20 bp, synthetic construct
  source 1..20: mol_type = other DNA
ccaatctactcagtaacact

SEQ ID NO: 36 - DNA, 20 bp, synthetic construct
  source 1..20: mol_type = other DNA
catctaaaatcggggttttt

SEQ ID NO: 37 - DNA, 20 bp, synthetic construct
  source 1..20: mol_type = other DNA
acacacctaagttgtgaaaa

SEQ ID NO: 38 - DNA, 20 bp, synthetic construct
  source 1..20: mol_type = other DNA
ctctcctaaactgcccctag

SEQ ID NO: 39 - DNA, 20 bp, synthetic construct
  source 1..20: mol_type = other DNA
tgaatctagctcactagctc

SEQ ID NO: 40 - DNA, 20 bp, synthetic construct
  source 1..20: mol_type = other DNA
actgccactagtctgtaggg

SEQ ID NO: 41 - DNA, 20 bp, synthetic construct
  source 1..20: mol_type = other DNA
tagaatctataattctgttg

SEQ ID NO: 42 - DNA, 20 bp, synthetic construct
  source 1..20: mol_type = other DNA
agtctagattggtttaatct

SEQ ID NO: 43 - DNA, 20 bp, synthetic construct
  source 1..20: mol_type = other DNA
gaagctaggagttcaagact

SEQ ID NO: 44 - DNA, 20 bp, synthetic construct
  source 1..20: mol_type = other DNA
gcctggctacatcatggcag

SEQ ID NO: 45 - DNA, 20 bp, synthetic construct
  source 1..20: mol_type = other DNA
atttctagttggagacactt

SEQ ID NO: 46 - DNA, 20 bp, synthetic construct
  source 1..20: mol_type = other DNA
gcactctagcctgaacagag

SEQ ID NO: 47 - DNA, 20 bp, synthetic construct
  source 1..20: mol_type = other DNA
gtagctgctatctcagaggc

SEQ ID NO: 48 - DNA, 20 bp, synthetic construct
  source 1..20: mol_type = other DNA
tactgtctaatttttcttct

SEQ ID NO: 49 - DNA, 20 bp, synthetic construct
  source 1..20: mol_type = other DNA
ctccgtctaaggagctggaa

SEQ ID NO: 50 - DNA, 20 bp, synthetic construct
  source 1..20: mol_type = other DNA
tcccgcctagaagcgcagcc

SEQ ID NO: 51 - DNA, 20 bp, synthetic construct
  source 1..20: mol_type = other DNA
cctgtctatggcttaggatc

SEQ ID NO: 52 - DNA, 20 bp, synthetic construct
  source 1..20: mol_type = other DNA
ctttatctattttgcagtgt

SEQ ID NO: 53 - DNA, 20 bp, synthetic construct
  source 1..20: mol_type = other DNA
caggggctacagggcatcca

SEQ ID NO: 54 - DNA, 20 bp, synthetic construct
  source 1..20: mol_type = other DNA
tggtcatctatttccaggtg

SEQ ID NO: 55 - DNA, 20 bp, synthetic construct
  source 1..20: mol_type = other DNA
catgcctattctgcaggtgt

SEQ ID NO: 56 - DNA, 20 bp, synthetic construct
  source 1..20: mol_type = other DNA
gactacctagtagtcatcaa

SEQ ID NO: 57 - DNA, 20 bp, synthetic construct
  source 1..20: mol_type = other DNA
aggctaaggcgggcggatca

SEQ ID NO: 58 - DNA, 20 bp, synthetic construct
  source 1..20: mol_type = other DNA
agactggctacacaaactgt

SEQ ID NO: 59 - DNA, 20 bp, synthetic construct
  source 1..20: mol_type = other DNA
gtcttctatttcctttcatt

SEQ ID NO: 60 - DNA, 20 bp, synthetic construct
  source 1..20: mol_type = other DNA
ctgttctatgggtggtcaga

SEQ ID NO: 61 - DNA, 20 bp, synthetic construct
  source 1..20: mol_type = other DNA
cacctctagcatctcagctc

SEQ ID NO: 62 - DNA, 20 bp, synthetic construct
  source 1..20: mol_type = other DNA
ctgggtctagttcagctgac

SEQ ID NO: 63 - DNA, 20 bp, synthetic construct
  source 1..20: mol_type = other DNA
aaagtctagaggcagaaggc

SEQ ID NO: 64 - DNA, 20 bp, synthetic construct
  source 1..20: mol_type = other DNA
ccagcctaggtgacagagca

SEQ ID NO: 65 - DNA, 20 bp, synthetic construct
  source 1..20: mol_type = other DNA
gcccctaaaaggcaaagcta

SEQ ID NO: 66 - DNA, 20 bp, synthetic construct
  source 1..20: mol_type = other DNA
ctgctctaactggccaccgg

SEQ ID NO: 67 - DNA, 20 bp, synthetic construct
  source 1..20: mol_type = other DNA
gtgagctatcgaaacaacct

SEQ ID NO: 68 - DNA, 20 bp, synthetic construct
  source 1..20: mol_type = other DNA
cagcatctacgagaagttct

SEQ ID NO: 69 - DNA, 20 bp, synthetic construct
  source 1..20: mol_type = other DNA
agtcgactaaggcagaattt

SEQ ID NO: 70 - DNA, 20 bp, synthetic construct
  source 1..20: mol_type = other DNA
cactgccctatctttgcata

SEQ ID NO: 71 - DNA, 20 bp, synthetic construct
  source 1..20: mol_type = other DNA
caaaatctattttccttcct

SEQ ID NO: 72 - DNA, 20 bp, synthetic construct
  source 1..20: mol_type = other DNA
gcgtctaggtaatcttcctc

SEQ ID NO: 73 - DNA, 20 bp, synthetic construct
  source 1..20: mol_type = other DNA
cagcgctatttatacctgga

SEQ ID NO: 74 - DNA, 20 bp, synthetic construct
  source 1..20: mol_type = other DNA
tgggggctactgccggcagg

SEQ ID NO: 75 - DNA, 20 bp, synthetic construct
  source 1..20: mol_type = other DNA
acatgctagaagtctgctgt

SEQ ID NO: 76 - DNA, 20 bp, synthetic construct
  source 1..20: mol_type = other DNA
tttggactatgtgacaaggg

SEQ ID NO: 77 - DNA, 20 bp, synthetic construct
  source 1..20: mol_type = other DNA
tgccactagtcctctagcac

SEQ ID NO: 78 - DNA, 20 bp, synthetic construct
  source 1..20: mol_type = other DNA
ggcctacaggctgtagaact

SEQ ID NO: 79 - DNA, 20 bp, synthetic construct
  source 1..20: mol_type = other DNA
ttcactatttcttccgcttc

SEQ ID NO: 80 - DNA, 20 bp, synthetic construct
  source 1..20: mol_type = other DNA
ccagctataaaaggcatgaa

SEQ ID NO: 81 - DNA, 20 bp, synthetic construct
  source 1..20: mol_type = other DNA
cgtttctactcatcggatcc

SEQ ID NO: 82 - DNA, 20 bp, synthetic construct
  source 1..20: mol_type = other DNA
gtgtgccctaggcgtagtat

SEQ ID NO: 83 - DNA, 20 bp, synthetic construct
  source 1..20: mol_type = other DNA
acactcactacacacgtttg

SEQ ID NO: 84 - DNA, 20 bp, synthetic construct
  source 1..20: mol_type = other DNA
ttggctaatggatccgagtt

SEQ ID NO: 85 - DNA, 20 bp, synthetic construct
  source 1..20: mol_type = other DNA
caagtctagttctgtggtgg

SEQ ID NO: 86 - DNA, 20 bp, synthetic construct
  source 1..20: mol_type = other DNA
tcagctctacactctcgtag

SEQ ID NO: 87 - DNA, 20 bp, synthetic construct
  source 1..20: mol_type = other DNA
tgatctctacttcagatcca

SEQ ID NO: 88 - DNA, 20 bp, synthetic construct
  source 1..20: mol_type = other DNA
aaggctacgtcccctgtcga

SEQ ID NO: 89 - DNA, 20 bp, synthetic construct
  source 1..20: mol_type = other DNA
gacttcctaggatctgtgcc

SEQ ID NO: 90 - DNA, 20 bp, synthetic construct
  source 1..20: mol_type = other DNA
aagcaggctaccttctccac

SEQ ID NO: 91 - DNA, 20 bp, synthetic construct
  source 1..20: mol_type = other DNA
gctggctaatggcccagctg

SEQ ID NO: 92 - DNA, 20 bp, synthetic construct
  source 1..20: mol_type = other DNA
gccttccctacacccaccct

SEQ ID NO: 93 - DNA, 20 bp, synthetic construct
  source 1..20: mol_type = other DNA
ccccagcctaggccgccctc

SEQ ID NO: 94 - DNA, 20 bp, synthetic construct
  source 1..20: mol_type = other DNA
aagagcctaggcagagagaa

SEQ ID NO: 95 - DNA, 20 bp, synthetic construct
  source 1..20: mol_type = other DNA
cttctactgggatacagcct

SEQ ID NO: 96 - DNA, 20 bp, synthetic construct
  source 1..20: mol_type = other DNA
gattaactacattcttacag

SEQ ID NO: 97 - DNA, 20 bp, synthetic construct
  source 1..20: mol_type = other DNA
gcccatctatcctgacctgt

SEQ ID NO: 98 - DNA, 20 bp, synthetic construct
  source 1..20: mol_type = other DNA
ttcctcctaaagatccagga

SEQ ID NO: 99 - DNA, 20 bp, synthetic construct
  source 1..20: mol_type = other DNA
gagtgctactgaaagtcgaa

SEQ ID NO: 100 - DNA, 20 bp, synthetic construct
  source 1..20: mol_type = other DNA
ttggaccctaggtagttttc

SEQ ID NO: 101 - DNA, 20 bp, synthetic construct
  source 1..20: mol_type = other DNA
gtcctagaggtggatccact

SEQ ID NO: 102 - DNA, 20 bp, synthetic construct
  source 1..20: mol_type = other DNA
cagctacaggcgcagcctct

SEQ ID NO: 103 - DNA, 20 bp, synthetic construct
  source 1..20: mol_type = other DNA
ctgtaggctaacgtggctgg

SEQ ID NO: 104 - DNA, 20 bp, synthetic construct
  source 1..20: mol_type = other DNA
ttctctagaagtcctgagac

SEQ ID NO: 105 - DNA, 20 bp, synthetic construct
  source 1..20: mol_type = other DNA
atccctagaaaaagaatccc

SEQ ID NO: 106 - DNA, 20 bp, synthetic construct
  source 1..20: mol_type = other DNA
aaaccctatgttgtagccac

SEQ ID NO: 107 - DNA, 20 bp, synthetic construct
  source 1..20: mol_type = other DNA
agctctaaggagcttgaaga

SEQ ID NO: 108 - DNA, 20 bp, synthetic construct
  source 1..20: mol_type = other DNA
agatgctagacttgcgtcag

SEQ ID NO: 109 - DNA, 20 bp, synthetic construct
  source 1..20: mol_type = other DNA
gagggtctactttgtggaga

SEQ ID NO: 110 - DNA, 20 bp, synthetic construct
  source 1..20: mol_type = other DNA
gtcttctactccaaaaactc

SEQ ID NO: 111 - DNA, 20 bp, synthetic construct
  source 1..20: mol_type = other DNA
ctcacctatgtcagtttctt

SEQ ID NO: 112 - DNA, 20 bp, synthetic construct
  source 1..20: mol_type = other DNA
ggccccctaccgatccatct

SEQ ID NO: 113 - DNA, 20 bp, synthetic construct
  source 1..20: mol_type = other DNA
gcatctaaaaccgcagtttc

SEQ ID NO: 114 - DNA, 20 bp, synthetic construct
  source 1..20: mol_type = other DNA
tccctgctagaagaacttga

SEQ ID NO: 115 - DNA, 20 bp, synthetic construct
  source 1..20: mol_type = other DNA
gctggctacgagtccggaac

SEQ ID NO: 116 - DNA, 20 bp, synthetic construct
  source 1..20: mol_type = other DNA
ccgcctctaccagaagtccc

SEQ ID NO: 117 - DNA, 20 bp, synthetic construct
  source 1..20: mol_type = other DNA
gaggcctagtcgagcaggga

SEQ ID NO: 118 - DNA, 20 bp, synthetic construct
  source 1..20: mol_type = other DNA
tcgctaggacagacggatcc

SEQ ID NO: 119 - DNA, 20 bp, synthetic construct
  source 1..20: mol_type = other DNA
tctgcctaatgtcggtaatg

SEQ ID NO: 120 - DNA, 20 bp, synthetic construct
  source 1..20: mol_type = other DNA
gtcagctaccagcagggtcc

SEQ ID NO: 121 - DNA, 20 bp, synthetic construct
  source 1..20: mol_type = other DNA
gtgattctaacagcggagcc

SEQ ID NO: 122 - DNA, 20 bp, synthetic construct
  source 1..20: mol_type = other DNA
tgtggtctagtgtgactttg

SEQ ID NO: 123 - DNA, 20 bp, synthetic construct
  source 1..20: mol_type = other DNA
ttgttctaatgcttcttgtt

SEQ ID NO: 124 - DNA, 20 bp, synthetic construct
  source 1..20: mol_type = other DNA
tgcacctagaagaaggtgtt

SEQ ID NO: 125 - DNA, 20 bp, synthetic construct
  source 1..20: mol_type = other DNA
gcggctagggccaagcctgc

SEQ ID NO: 126 - DNA, 20 bp, synthetic construct
  source 1..20: mol_type = other DNA
cggcccctacttgccggcga

SEQ ID NO: 127 - DNA, 20 bp, synthetic construct
  source 1..20: mol_type = other DNA
ggggccctaggagggggccc

SEQ ID NO: 128 - DNA, 20 bp, synthetic construct
  source 1..20: mol_type = other DNA
gccaaccctatttcaggcag

SEQ ID NO: 129 - DNA, 20 bp, synthetic construct
  source 1..20: mol_type = other DNA
ggacactactggcaaaaggg

SEQ ID NO: 130 - DNA, 20 bp, synthetic construct
  source 1..20: mol_type = other DNA
atggactagtgtctcggctt

SEQ ID NO: 131 - DNA, 20 bp, synthetic construct
  source 1..20: mol_type = other DNA
ggtctctatggtgggagcac

SEQ ID NO: 132 - DNA, 20 bp, synthetic construct
  source 1..20: mol_type = other DNA
ggccgcctacaatttgtcca

SEQ ID NO: 133 - DNA, 20 bp, synthetic construct
  source 1..20: mol_type = other DNA
ttctcatctattgataataa

SEQ ID NO: 134 - DNA, 20 bp, synthetic construct
  source 1..20: mol_type = other DNA
agccactacttcttattcct

SEQ ID NO: 135 - DNA, 20 bp, synthetic construct
  source 1..20: mol_type = other DNA
aggctctaagcactgccagg

SEQ ID NO: 136 - DNA, 20 bp, synthetic construct
  source 1..20: mol_type = other DNA
aggttctaggagaagatgga

SEQ ID NO: 137 - DNA, 20 bp, synthetic construct
  source 1..20: mol_type = other DNA
cttctagtgttgggtccact

SEQ ID NO: 138 - DNA, 20 bp, synthetic construct
  source 1..20: mol_type = other DNA
atttgctaccaccaaaatct

SEQ ID NO: 139 - DNA, 20 bp, synthetic construct
  source 1..20: mol_type = other DNA
tcttgtctacttcttcctcc

SEQ ID NO: 140 - DNA, 20 bp, synthetic construct
  source 1..20: mol_type = other DNA
ctgctgctaccaggagctga

SEQ ID NO: 141 - DNA, 20 bp, synthetic construct
  source 1..20: mol_type = other DNA
acctctagtggtcatcaggc

SEQ ID NO: 142 - DNA, 20 bp, synthetic construct
  source 1..20: mol_type = other DNA
tgtctctagtgtatgtctag

SEQ ID NO: 143 - DNA, 20 bp, synthetic construct
  source 1..20: mol_type = other DNA
tgagtctagaccttgtacag

SEQ ID NO: 144 - DNA, 20 bp, synthetic construct
  source 1..20: mol_type = other DNA
gggctagttgctggtccacc

SEQ ID NO: 145 - DNA, 20 bp, synthetic construct
  source 1..20: mol_type = other DNA
caactctacagacataattc

SEQ ID NO: 146 - DNA, 20 bp, synthetic construct
  source 1..20: mol_type = other DNA
atactgctactgctcattgg

SEQ ID NO: 147 - DNA, 20 bp, synthetic construct
  source 1..20: mol_type = other DNA
aagtactaaatattgctgag

SEQ ID NO: 148 - DNA, 20 bp, synthetic construct
  source 1..20: mol_type = other DNA
ttatctactgtgaatcaatc

SEQ ID NO: 149 - DNA, 20 bp, synthetic construct
  source 1..20: mol_type = other DNA
ttgtttctagtatctgcttc

SEQ ID NO: 150 - DNA, 20 bp, synthetic construct
  source 1..20: mol_type = other DNA
ggaatctacggctccagctc

SEQ ID NO: 151 - DNA, 23254 bp, synthetic construct
  source 1..23254: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgccaccatggacaacaccgaggacgtcatcaaggagttcatgcagttcaaggtgcgcatggagggctccgtgaacggccactacttcgagatcgagggcgagggcgagggcaagccctacgagggcacccagaccgccaagctgcaggtgaccaagggcggccccctgcccttcgcctgggacatcctgtccccccagttccagtacggctccaaggcctacgtgaagcaccccgccgacatccccgactacatgaagctgtccttccccgagggcttcacctgggagcgctccatgaacttcgaggacggcggcgtggtggaggtgcagcaggactcctccctgcaggacggcaccttcatctacaaggtgaagttcaagggcgtgaacttccccgccgacggccccgtaatgcagaagaaaactgccggctgggagccctccaccgagaagctgtacccccaggacggcgtgctgaagggcgagatctcccacgccctgaagctgaaggacggcggccactacacctgcgacttcaagaccgtgtacaaggccaagaagcccgtgcagctgcccggcaaccactacgtggactccaagctggacatcaccaaccacaacgaggactacaccgtggtggagcagtacgagcacgccgaggcccgccactccggctcccagtaagcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtcgacattgattattgactagttgccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccaaacctagcctattatccgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagctctaataaaccgagcacgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccctcctagcccgacgtgacgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggccctaggtgaggatgtcagttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccatctaagatagcagcagcgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagcaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcctagctacttgggagtctggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtctctagagatggtttatcagttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagaatctctatgtcttttgggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtttggctacttggtctcttcgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggatgcttctagaagcctggagttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaactagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgttcgtccctagctctgaagggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcctttcctaagacattgctagttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtggaggctactccagaaccagttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggaaaagctatactctagtgggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgtcctacagctttgtccaggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagattacgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgttcagttctaagtcaacgttgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatctcctaattcttcacaaagttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgcctactcattttcttcacgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatggtggctatccacgatgggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatagctaaacactcatcattgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagatgtggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggccaactatggcgtgacagagttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtcattctatttgccttttttgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggctttctagcagcctccccagttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgttgtgctacagactaaatccgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacaactaaagttctgagctagttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggattcctaagactgtttgctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcttttcctagtgcccataccgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcaaggctaagtcccacgtgcgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcaatcttctatttctctaaagttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtcctcgctacaggaagctgcgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaaggagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtctttcctatttcttcacacgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggaaacagctacaaaaccagtgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagctctacgtcagcttccagttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggggctagttgaatttagcctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccaatctactcagtaacactgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaattcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcatctaaaatcggggtttttgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacacacctaagttgtgaaaagttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctctcctaaactgcccctaggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgaatctagctcactagctcgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtagctgctatctcagaggcgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtactgtctaatttttcttctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctccgtctaaggagctggaagttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtcccgcctagaagcgcagccgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagacagaactagtaatagtaatcaattacggggtcattagttcatagcccatatatggagttccgaatagtaatcaattacggggtcattagttcatagcccatatatggagttccgaatagtaatcaattacggggtcattagttcatagcccatatatggagttccg

(19) United States
(12) Patent Application Publication
(10) Pub. No.: US 2024/0368588 A1
(43) Pub. Date: Nov. 7, 2024

(54) METHOD FOR HIGH-THROUGHPUT TAG to TAA CONVERSION ON GENOME

(71) Applicants: SHENZHEN INSTITUTES OF ADVANCED TECHNOLOGY, Shenzhen (CN); SHENZHEN INSTITUTE OF ADVANCED TECHNOLOGY CHINESE ACADEMY OF SCIENCES, Shenzhen (CN)

(72) Inventor: Yuting CHEN, Shenzhen (CN)

(73) Assignees: SHENZHEN INSTITUTES OF ADVANCED TECHNOLOGY, Shenzhen (CN); SHENZHEN INSTITUTE OF ADVANCED TECHNOLOGY CHINESE ACADEMY OF SCIENCES, Shenzhen (CN)

(21) Appl. No.: 18/621,103

(22) Filed: Mar. 29, 2024

Related U.S. Application Data
(63) Continuation of application No. PCT/CN2021/121750, filed on Sep. 29, 2021.

(51) Int. Cl. C12N 15/11; C12N 9/22; C12N 15/85; C12Q 1/6874

(52) U.S. Cl. CPC C12N 15/111; C12N 9/22; C12N 15/85; C12Q 1/6874; C12N 2310/20

(57) ABSTRACT

A method for high-throughput TAG to TAA conversion on the genome are provided. The method comprises the following steps: co-transfecting a gRNA array pool or a transcription product thereof, a plasmid containing an mCherry-inactivated eGFP reporter molecule and an sgRNA plasmid for editing and activating eGFP in a stable cell of an inducible base editor; or by transfecting an 43-all-in-one expression vector or a transcription product thereof to cells with stable inducible base editor, high-flux TAG to TAA conversion in single cells is realized, and through multiple cyclic operations, almost all TAG to TAA conversion in the whole genome of cells of common model organisms can be realized.

### SEQUENCE LISTING

The sequence listing xml file submitted herewith, named “SEQUENCE_LISTING.xml”, created on Jun. 17, 2024, and having a file size of 157,403 bytes, is incorporated by reference herein.

### TECHNICAL FIELD

The present invention relates to the field of biotechnology, and particularly to a method for high-throughput TAG to TAA conversion on the genome.

### BACKGROUND

The genetic code has degeneracy in that except for the 3 stop triplet codons for terminating the translation, the other 61 triplet codons encode 20 natural amino acids, and thus, 18 out of the 20 amino acids are encoded by more than one synonymous codon. Recoding is a promising application of genome engineering. It involves replacing all specific codons in the genome with synonymous codons and knocking out the corresponding transfer RNA (tRNA), such that the recoded cells possess the same proteome as before, but use a simplified genetic code. Recoding can impart cells with viral resistance, or impart “blank” codons with new functionality, including nonstandard amino acid integration and biological protection.

The first whole genome recoding was reported by Church Lab, in which 314 UAG stop codons in Escherichia coli were substituted with UAA. All UAG to UAA substitutions and the deletion of release factor 1 (which allows the termination of translation by UAG and UAA) were then tested in E. coli, and reduced infectivity of 4 viruses (λ, M13, P1, MS2) was observed in E. coli. In another study, 13 sense codons on a set of ribosomal genes were modified and 123 instances of two rare arginine codons were synonymously substituted. Recently, Church Lab synthesized and assembled an E. coli genome with 3.97 million bases and 57 codons, and Jason Chin's laboratory has completed the complete recoding and assembly of an E. coli strain with 61 codons and deleted the tRNAs and release factor 1, which resulted in complete resistance to virus cocktails in the cells. These codons were used for the efficient synthesis of proteins containing three different non-standard amino acids in SYN61. However, no reprogramming in mammalian cells, especially in the human genome, has yet been reported.

The CRISPR-Cas technology enhances the capability of modifying genomes, and can edit specific genes or regulate the transcription thereof by designing guide RNAs (gRNAs). More precise tools, such as base editors, guide editors, transposons, integrons, etc., were subsequently derived from CRISPR-Cas. Although CRISPR-Cas and its derivative tools have good universality, the use of individual gRNAs limits their efficiency and applications in biotechnology: Thus, multiplexed strategies are used in an increasing number of studies for multi-site editing or transcriptional regulation. Multiplexed CRISPR refers to a technique for greatly improving the range and efficiency of gene editing and transcriptional regulation by the expression of many gRNAs or Cas enzymes to promote bioengineering applications. Currently, two main approaches have been presented to express multiple gRNAs in individual cells. One is to transcribe each gRNA expression cassette with a single RNA polymerase promoter and then clone multiple gRNA expression cassettes into a single plasmid by Golden gate assembly. The other approach is to transcribe all gRNAs into one transcript by using one promoter and then treat to release individual gRNAs by different strategies that require cleavable RNA sequences at ends of each gRNA, such as self-cleaving ribozyme sequences (e.g., hammerhead ribozyme and HDV ribozyme), exogenous cleavage factor recognition sequences (e.g., Cys4), and endogenous RNA processing sequences (e.g., tRNA sequences and introns).

Single TAG to TAA conversions can be achieved in individual cells by transfecting the cells with sgRNAs and CBEs targeting the site. However, if tens or hundreds of TAG to TAA conversions are required in a single cell, it may require to convey as many corresponding sgRNAs and CBEs as possible in one delivery: No tools are currently available for this application.

Therefore, it is of great interest to develop a technique that achieves high-throughput TAG to TAA conversion in individual cells.

### SUMMARY

In order to solve the technical problems in the prior art, the present invention is intended to provide a method for high-throughput TAG to TAA conversion on the genome. The specific solution is as follows:

In a first aspect, the present invention provides a gRNA array, comprising 5 sgRNA expression cassettes connected in series, wherein each sgRNA expression cassette comprises a promoter, an sgRNA and a poly T in the 5′ to 3′ direction; the sgRNA in the sgRNA expression cassette is selected from the sequence set forth in one of SEQ ID NOs. 1-150, and the sgRNAs of the gRNA array are different from each other.

Preferably, the 5 sgRNA expression cassettes connected in series are chemically synthesized.

In a second aspect, the present invention provides a gRNA array pool, comprising 2-10 gRNA arrays, wherein each gRNA array comprises 5 sgRNA expression cassettes connected in series, wherein each sgRNA expression cassette comprises a promoter, an sgRNA and a polyT in the 5′ to 3′ direction; the sgRNA in the sgRNA expression cassette is selected from the sequence set forth in one of SEQ ID NOs. 1-150, and the sgRNAs of the gRNA array pool are different from each other: preferably, the gRNA array pool comprises 10 gRNA arrays.

Preferably, the 5 sgRNA expression cassettes connected in series are chemically synthesized.

In a third aspect, the present invention provides an expression vector having a nucleotide sequence set forth in SEQ ID NO. 151.

In a fourth aspect, the present invention provides a bacterium comprising the expression vector.

In a fifth aspect, the present invention provides a base editing system comprising the gRNA array pool or a transcript thereof, or the expression vector or a transcript thereof.

The base editing system further comprises a base editor, wherein the base editor is selected from an adenine base editor or a cytosine base editor;
preferably, the base editor is a cytosine base editor.

In a sixth aspect, the present invention provides a kit for multiplex base editing comprising the base editing system;
preferably, the kit further comprises a plasmid containing an mCherry-inactivated eGFP reporter and an sgRNA plasmid for editing and activating eGFP.

In a seventh aspect, the present invention provides a method for high-throughput TAG to TAA conversion on the genome, comprising:
transfecting a cell with a gRNA array by the following method to achieve TAG to TAA conversion:
I: co-transfecting the gRNA array pool or a transcript thereof, a plasmid containing an mCherry-inactivated eGFP reporter, an sgRNA plasmid for editing and activating eGFP, and a base editor into the cell: or
II: co-transfecting the expression vector or a transcript thereof and a base editor into the cell.

In an eighth aspect, the present invention provides a method for high-throughput TAG to TAA conversion on the genome, comprising:
transfecting a cell with a gRNA array by the following method to achieve TAG to TAA conversion:
I: co-transfecting the gRNA array pool or a transcript thereof, a plasmid containing an mCherry-inactivated eGFP reporter, and an sgRNA plasmid for editing and activating eGFP into a cell having a stable inducible base editor: or
II: transfecting the expression vector or a transcript thereof into a cell having a stable inducible base editor.

The method for high-throughput TAG to TAA conversion on genome further comprises: isolating monoclones from the transfected cells and culturing, performing Sanger sequencing and EditR analysis, selecting monoclones with high editing efficiency, and transfecting with a gRNA array by method I or II, preferably method I.

According to the method for high-throughput TAG to TAA conversion on genome, the cell is a mammalian cell; preferably, the mammalian cell is a human mammalian cell.

According to the method for high-throughput TAG to TAA conversion on genome, in I, as per 1×10₅ mammalian cells, the transfection amount of the gRNA array is 200 ng, the transfection amount of the plasmid containing an mCherry-inactivated eGFP reporter is 30 ng, and the transfection amount of the sgRNA plasmid for editing and activating eGFP is 10 ng;
in II, as per 1×10⁵ mammalian cells, the transfection amount of the expression vector is 2 μg.

According to the method for high-throughput TAG to TAA conversion on genome, the cell having a stable inducible base editor is selected from a cell monoclone having a stable inducible base editor with high editing efficiency.

Further, the method for screening the cell monoclone having a stable inducible base editor with high editing efficiency comprises: selecting cell monoclones having a stable inducible base editor denoted as original monoclones; and transfecting one gRNA array into the selected original monoclones, and selecting transfected monoclones with high editing efficiency, wherein the original monoclones corresponding to the transfected monoclones with high editing efficiency are the cell monoclones having a stable inducible base editor with high editing efficiency.

Further, the inducible base editor is a doxycycline-inducible base editor, preferably a doxycycline-inducible cytosine base editor;

preferably, the cell having a stable inducible base editor is selected from a mammalian cell stably expressing PB-FNLS-BE3-NG1 or PB-evoAPOBEC1-BE4max-NG.

In a ninth aspect, the present invention provides a cell edited by the method for high-throughput TAG to TAA conversion on genome.

The present invention has the following beneficial effects:
1. The method for high-throughput TAG to TAA conversion on genome of the present invention achieves high-throughput TAG to TAA conversion in individual cells by co-transfecting the gRNA array pool or a transcript thereof, a plasmid containing an mCherry-inactivated eGFP reporter, and an sgRNA plasmid for editing and activating eGFP into a cell having a stable inducible base editor or by transfecting an 43-all-in-one expression vector or a transcript thereof into a cell having a stable inducible base editor, and achieve almost all TAG to TAA conversions in the whole genome via multiple cycles.
2. According to the present invention, gBlocks or 43-all-in-one expression vector is transfected into a mammal cell with a stable inducible base editor, such that stable and continuous expression of the base editor can be achieved with the induction of doxycycline, resulting in higher base editing efficiency than that of transient expression. As a preferable embodiment, the base editing efficiency can be further improved by selecting the mammalian cell monoclone having a stable inducible base editor with high editing efficiency and further transfecting gBlocks or 43-all-in-one expression vector into the selected monoclones with high editing efficiency.
3. As a preferred embodiment, the present invention co-transfects gBlocks with a plasmid containing an mCherry-inactivated eGFP reporter and an sgRNA plasmid for editing and activating eGFP into mammalian cells, with the amount of transfected reporter being about one tenth of that of each gBlocks. When both the reporter and the corresponding sgRNA are transfected into individual cells simultaneously, the number of sgRNAs transfected into the targeted gene loci in the individual cells of the gBlock may be greater. When the reporter and the corresponding sgRNA are in a single cell and the single base editing occurs, cells with green fluorescence and cells with red and green dual fluorescence can be detected, indicating that a greater amount of sgRNAs is transfected and the editing occurs. Enrichment of high-editing clones can be achieved by flow cytometric sorting.

### BRIEF DESCRIPTION OF DRAWINGS

FIG. 1 a structural schematic of gBlock-YC1 and gBlock PC in Example 2.

FIG. 2 the results of the base editing efficiency verification in target loci in Example 2, wherein FIG. 2-a shows the editing efficiency of gBlock-PC, and FIG. 2-b shows the editing efficiency of gBlock-YC1: dots represent individual biological replicates and bars represent mean values.

FIG. 3 a structural schematic of doxycycline-inducible cytidine deaminase piggy Bac in Example 3, wherein F denotes the Flag tag: NLS denotes nuclear localization signal; cas9n-NG denotes a Cas9D10A recognizing NG-PAM: APOBEC1 denotes rat APOBEC1: evoAPOBEC1 denotes evolved rat APOBEC1.

FIG. 4 the results of the base editing efficiency verification in target loci in Example 3, wherein FIG. 4-a shows the editing efficiency of gBlock-PC, and FIG. 4-b shows the editing efficiency of gBlock-YC1: dots and triangles represent individual biological replicates and bars represent mean values.

FIG. 5 the protein level of cytosine base editor in transfected cell monoclones stably expressing evoAPOBEC1-BE4max-NG in Example 4, determined by using anti-Cas9 (upper) and anti-actin (lower).

FIG. 6 the results of the base editing efficiency verification in target loci in Example 4, wherein the values and error bars denote the mean and standard deviation of four independent measurements.

FIG. 7 a cell line stably expressing evoAPOBEC1-BE4max-NG introduced by a gBlocks pool in Example 5.

FIG. 8 a heatmap of target “C” editing efficiency based on whole exome sequencing in Example 5.

FIG. 9 a flowchart of the construction of integrative plasmid in Example 6.

FIG. 10 the agarose gel electrophoresis of the integrative plasmid in Example 6; wherein, the left lane was DNA ladders, and the rightmost empty vector was the control group: the arrows in lanes 5 and 7 were 22 Kb.

FIG. 11 basic quality attributes in single cell RNA sequencing with 3 different delivery methods in Example 7, wherein a denotes the number of cells captured, b denotes the number of UMIs per unit, and c denotes the number of genes detected per cell.

FIG. 12-13 the distribution analysis of target cells with different modified genes in populations with different delivery methods based on single cell RNAseq in Example 7, wherein, FIG. 12a illustrate the relationship between the number of edited gene loci and the number of cells in the 3 populations: FIG. 12b illustrates the distribution of edited gene loci detected by scRNAseq in the 3 populations with the vertical line denoting the median of edited gene loci; FIG. 12c, FIG. 13d and FIG. 13e illustrates the distribution analysis of modified cells with different editing efficiency for each gene locus as determined by different methods.

FIG. 14 the editing efficiency of sgRNA in single cells with different delivery modes by single cell sequencing analysis in Example 7, wherein g illustrates the editing efficiency of each sgRNA in single cells: h illustrates the heatmap of the editing efficiency of target C in the cell populations with the three delivery methods based on the conversion of single cell RNA-Seq to cell population RNA-Seq: the editing efficiency is shown in black intensity.

FIG. 15 a monoclone screen by Sanger sequencing in Example 8, wherein a, 10 well edited loci were selected, the peak number of gBlocks was 3, and only one clone had all of the 10 gBlocks: b, 3 well edited loci were selected for screening, half of the clones showed no edit, and 4 clones had all of 3 edited loci: c, all target loci were subjected to allelic editing by Sanger sequencing and EditR: WT (wild-type) denotes no allele editing: HZ (heterozygote) denotes partial allele editing; HM (homozygous) denotes all allele editing.

FIG. 16-19 the genetic variation analysis by WGS to identify highly modified HEK293T clones in Example 9, wherein FIG. 16a illustrates the efficiency of TAG to TAA conversion by heatmap editing of target “C”, in which the columns are sequentially the NC-negative control, clone 19 in method 2, clone 21 in method 3, clones 19-1, 19-16 and 19-21 obtained by secondary transfection using method 2 on the basis of clone 19, and the number of exon SNVs (SNVs located in exons and splice sites) or other SNVs detected in the highly modified clones compared to the sequence of the parent HEK293T: the total SNV numbers of clone 19, clone 21, clone 19-1, clone 19-16, and clone 19-21 compared to the sequence of the parent HEK293T were 23084, 70356, 35700, 42595, and 31530, respectively: FIG. 17c illustrates the number of exon SNVs detected in essential genes: FIG. 17d illustrates the distribution of different types of SNV variation: FIG. 17e illustrates the mutation rate of C>T or G>T SNVs detected among the samples; FIG. 18f illustrates the mutation rate of C>T or G>TSNVs detected among samples and chromosomes; FIG. 19g illustrates the number of exon indels or other indels detected in highly modified clones; FIG. 19h illustrates the mutation rate of indels detected in the sample; i illustrates the mutation rate of indels detected among samples and chromosomes.

FIG. 20-21 the chromosomal distribution of exon SNV in essential genes in Example 9, wherein, FIG. 20a contains 50 selected essential gene targets while FIG. 21b does not: the X-axis represents the chromosomes and the y-axis represents the count in chromosomes; for better display, the number of exon SNVs of essential genes on each chromosome is marked at the top of each bar.

### DETAILED DESCRIPTION OF THE EMBODIMENTS

In order to understand the present invention more clearly, the present invention will be further described with reference to the following examples and drawings. The examples are given for the purpose of illustration only and are not intended to limit the present invention in any way. In the examples, all of the reagents and starting materials are commercially available, and the experimental methods without specifying the specific conditions are conventional methods with conventional conditions well known in the art, or conditions suggested by the instrument manufacturer.

The single base editing system is a base editing system combining CRISPR/Cas9 and cytosine deaminase. With the system, a fusion protein formed by Cas9-cytosine deaminase-uracil glycosylase inhibitor can target a specific locus complementary to gRNA (a sequence complementary to the target DNA in the sgRNA) by using the sgRNA without breaking the double-stranded DNA, and the amino group of cytosine (C) at the target locus can be removed, such that C is converted into uracil (U). Along with the replication of DNA, the U is replaced by thymine (T), and finally, the single base mutation of C→T is achieved.

CBE denotes cytosine base editor. Rat APOBEC1 (rAPOBEC1) is present in the widely used CBE editors, BE3 and BE4. rAPOBEC1 enzyme induces the deamination of cytosine (C) in DNA and is directed by Cas protein and gRNA complexes to the specific target loci. evoAPOBEC1 denotes evolved APOBEC1.

### Example 1

In one embodiment of the present invention, a gRNA array is provided, comprising 5 sgRNA expression cassettes connected in series, wherein each sgRNA expression cassette comprises a promoter, an sgRNA and a poly T in the 5′ to 3′ direction; the sgRNA in the sgRNA expression cassette is selected from any nucleotide sequence set forth in SEQ ID NOs. 1-150 (Table 1), and the sgRNAs of the gRNA array are different from each other. As a preferred embodiment, the 5 sgRNA expression cassettes connected in series are chemically synthesized.

In one embodiment of the present invention, a gRNA array pool is provided, comprising 2-10 gRNA arrays, wherein each gRNA array comprises 5 sgRNA expression cassettes connected in series, wherein each sgRNA expression cassette comprises a promoter, an sgRNA and a polyT in the 5′ to 3′ direction; the sgRNA in the sgRNA expression cassette is selected from any nucleotide sequence set forth in SEQ ID NOs. 1-150 (Table 1), and the sgRNAs of the gRNA array are different from each other. As a preferred embodiment, the 5 sgRNA expression cassettes connected in series are chemically synthesized. A greater amount of gRNA arrays transfected into the cell may achieve a higher base editing efficiency. In a preferred embodiment of the present invention, the gRNA array pool comprises 10 gRNA arrays.

Table 1 shows 150 sgRNAs targeting 152 loci. The same gene in Table 1 indicates that the sgRNA sequence targets two positions, and loci No. 10, 12, and 13 are targeted by the same sgRNA sequence.

TABLE 1
150 sgRNAs targeting 152 loci

| | | | SEQ |
|---|---|---|---|
| | Gene | | ID |
| No. | (position) | sgRNA sequence | NO |
| 1 | ORC3 | CCAAACCTAGCCTATTATCC | 1 |
| 2 | ORC3 | AGCTCTAATAAACCGAGCAC | 2 |
| 3 | PTPA | CCCTCCTAGCCCGACGTGAC | 3 |
| 4 | PSMD13 | GGCCCTAGGTGAGGATGTCA | 4 |
| 5 | NOP2 | CCATCTAAGATAGCAGCAGC | 5 |
| 6 | NOP2 | CCTAGCTACTTGGGAGTCTG | 6 |
| 7 | ANAPC5 | TCTCTAGAGATGGTTTATCA | 7 |
| 8 | KIAA0391 | AGAATCTCTATGTCTTTTGG | 8 |
| 9 | AQR | TTTGGCTACTTGGTCTCTTC | 9 |
| 10 | TBC1D3B | GATGCTTCTAGAAGCCTGGA | 10 |
| 11 | TBC1D3F | TTCGTCCCTAGCTCTGAAGG | 11 |
| 12 | TBC1D3C | GATGCTTCTAGAAGCCTGGA | 10 |
| 13 | TBC1D3 | GATGCTTCTAGAAGCCTGGA | 10 |
| 14 | BIRC5 | CCTTTCCTAAGACATTGCTA | 12 |
| 15 | MRPL12 | TGGAGGCTACTCCAGAACCA | 13 |
| 16 | NLGN4Y | GAAAAGCTATACTCTAGTGG | 14 |
| 17 | SRY | TGTCCTACAGCTTTGTCCAG | 15 |
| 18 | WDR3 | TTCAGTTCTAAGTCAACGTT | 16 |
| 19 | ECT2 | ATCTCCTAATTCTTCACAAA | 17 |
| 20 | RPL32 | TGCCTACTCATTTTCTTCAC | 18 |
| 21 | TFRC | ATGGTGGCTATCCACGATGG | 19 |
| 22 | POLR2B | ATAGCTAAACACTCATCATT | 20 |
| 23 | CDC23 | GCCAACTATGGCGTGACAGA | 21 |
| 24 | RIOK1 | TCATTCTATTTGCCTTTTTT | 22 |
| 25 | ORC3 | GCTTTCTAGCAGCCTCCCCA | 23 |
| 26 | MASTL | TTGTGCTACAGACTAAATCC | 24 |
| 27 | ATP2A2 | ACAACTAAAGTTCTGAGCTA | 25 |
| 28 | AURKA | GATTCCTAAGACTGTTTGCT | 26 |
| 29 | RBX1 | CTTTTCCTAGTGCCCATACC | 27 |
| 30 | LOC105373102 | CAAGGCTAAGTCCCACGTGC | 28 |
| 31 | CD99 | CAATCTTCTATTTCTCTAAA | 29 |
| 32 | ZBED1 | TCCTCGCTACAGGAAGCTGC | 30 |
| 33 | VAMP7 | TCTTTCCTATTTCTTCACAC | 31 |
| 34 | UTY | GAAACAGCTACAAAACCAGT | 32 |
| 35 | PPIE | GAGCTCTACGTCAGCTTCCA | 33 |
| 36 | NUDC | GGGCTAGTTGAATTTAGCCT | 34 |
| 37 | WDR77 | CCAATCTACTCAGTAACACT | 35 |
| 38 | SFPQ | CATCTAAAATCGGGGTTTTT | 36 |
| 39 | SFPQ | ACACACCTAAGTTGTGAAAA | 37 |
| 40 | NSL1 | CTCTCCTAAACTGCCCCTAG | 38 |
| 41 | RABGGTB | TGAATCTAGCTCACTAGCTC | 39 |
| 42 | ISG20L2 | ACTGCCACTAGTCTGTAGGG | 40 |
| 43 | DTL | TAGAATCTATAATTCTGTTG | 41 |
| 44 | MAGOH | AGTCTAGATTGGTTTAATCT | 42 |
| 45 | ZBTB8OS | GAAGCTAGGAGTTCAAGACT | 43 |
| 46 | TRNAU1AP | GCCTGGCTACATCATGGCAG | 44 |
| 47 | SNRPE | ATTTCTAGTTGGAGACACTT | 45 |
| 48 | MTOR | GCACTCTAGCCTGAACAGAG | 46 |
| 49 | POLRIA | GTAGCTGCTATCTCAGAGGC | 47 |
| 50 | ATL2 | TACTGTCTAATTTTTCTTCT | 48 |
| 51 | WDR33 | CTCCGTCTAAGGAGCTGGAA | 49 |
| 52 | UQCRC1 | TCCCGCCTAGAAGCGCAGCC | 50 |
| 53 | THOC7 | CCTGTCTATGGCTTAGGATC | 51 |
| 54 | PSMD6 | CTTTATCTATTTTGCAGTGT | 52 |
| 55 | RPN1 | CAGGGGCTACAGGGCATCCA | 53 |
| 56 | RUVBL1 | TGGTCATCTATTTCCAGGTG | 54 |
| 57 | FIP1L1 | CATGCCTATTCTGCAGGTGT | 55 |
| 58 | ETF1 | GACTACCTAGTAGTCATCAA | 56 |
| 59 | NSA2 | AGGCTAAGGCGGGCGGATCA | 57 |
| 60 | PRELID1 | AGACTGGCTACACAAACTGT | 58 |
| 61 | SRSF3 | GTCTTCTATTTCCTTTCATT | 59 |
| 62 | MDN1 | CTGTTCTATGGGTGGTCAGA | 60 |
| 63 | FARS2 | CACCTCTAGCATCTCAGCTC | 61 |
| 64 | RPL7L1 | CTGGGTCTAGTTCAGCTGAC | 62 |
| 65 | RARS2 | AAAGTCTAGAGGCAGAAGGC | 63 |
| 66 | VPS52 | CCAGCCTAGGTGACAGAGCA | 64 |
| 67 | WDR46 | GCCCCTAAAAGGCAAAGCTA | 65 |
| 68 | RFC2 | CTGCTCTAACTGGCCACCGG | 66 |
| 69 | TNPO3 | GTGAGCTATCGAAACAACCT | 67 |
| 70 | OGDH | CAGCATCTACGAGAAGTTCT | 68 |
| 71 | BUD31 | AGTCGACTAAGGCAGAATTT | 69 |
| 72 | NUP188 | CACTGCCCTATCTTTGCATA | 70 |
| 73 | SMC2 | CAAAATCTATTTTCCTTCCT | 71 |
| 74 | POLRIE | GCGTCTAGGTAATCTTCCTC | 72 |
| 75 | MED22 | CAGCGCTATTTATACCTGGA | 73 |
| 76 | MED27 | TGGGGGCTACTGCCGGCAGG | 74 |
| 77 | IARS | ACATGCTAGAAGTCTGCTGT | 75 |
| 78 | POLR3A | TTTGGACTATGTGACAAGGG | 76 |
| 79 | PDCD11 | TGCCACTAGTCCTCTAGCAC | 77 |
| 80 | PRPF19 | GGCCTACAGGCTGTAGAACT | 78 |
| 81 | NAT10 | TTCACTATTTCTTCCGCTTC | 79 |
| 82 | NARS2 | CCAGCTATAAAAGGCATGAA | 80 |
| 83 | SSRP1 | CGTTTCTACTCATCGGATCC | 81 |
| 84 | PSMC3 | GTGTGCCCTAGGCGTAGTAT | 82 |
| 85 | MRPL16 | ACACTCACTACACACGTTTG | 83 |
| 86 | DDB1 | TTGGCTAATGGATCCGAGTT | 84 |
| 87 | SF1 | CAAGTCTAGTTCTGTGGTGG | 85 |
| 88 | HINFP | TCAGCTCTACACTCTCGTAG | 86 |
| 89 | CLP1 | TGATCTCTACTTCAGATCCA | 87 |
| 90 | INTS5 | AAGGCTACGTCCCCTGTCGA | 88 |
| 91 | NCAPD2 | GACTTCCTAGGATCTGTGCC | 89 |
| 92 | RFC5 | AAGCAGGCTACCTTCTCCAC | 90 |
| 93 | POLE | GCTGGCTAATGGCCCAGCTG | 91 |
| 94 | POLE | GCCTTCCCTACACCCACCCT | 92 |
| 95 | DDX51 | CCCCAGCCTAGGCCGCCCTC | 93 |
| 96 | DDX51 | AAGAGCCTAGGCAGAGAGAA | 94 |
| 97 | RFC3 | CTTCTACTGGGATACAGCCT | 95 |
| 98 | POLE2 | GATTAACTACATTCTTACAG | 96 |
| 99 | PABPN1 | GCCCATCTATCCTGACCTGT | 97 |
| 100 | DLST | TTCCTCCTAAAGATCCAGGA | 98 |
| 101 | WARS | GAGTGCTACTGAAAGTCGAA | 99 |
| 102 | MFAP1 | TTGGACCCTAGGTAGTTTTC | 100 |
| 103 | GTF3C1 | GTCCTAGAGGTGGATCCACT | 101 |
| 104 | COG4 | CAGCTACAGGCGCAGCCTCT | 102 |
| 105 | NUBP1 | CTGTAGGCTAACGTGGCTGG | 103 |
| 106 | GINS2 | TTCTCTAGAAGTCCTGAGAC | 104 |
| 107 | RPS15A | ATCCCTAGAAAAAGAATCCC | 105 |
| 108 | RPS2 | AAACCCTATGTTGTAGCCAC | 106 |
| 109 | DCTN5 | AGCTCTAAGGAGCTTGAAGA | 107 |
| 110 | DCTN5 | AGATGCTAGACTTGCGTCAG | 108 |
| 111 | ATP6VOC | GAGGGTCTACTTTGTGGAGA | 109 |
| 112 | SMG6 | GTCTTCTACTCCAAAAACTC | 110 |
| 113 | PSMD11 | CTCACCTATGTCAGTTTCTT | 111 |
| 114 | SUPT6H | GGCCCCCTACCGATCCATCT | 112 |
| 115 | RPL27 | GCATCTAAAACCGCAGTTTC | 113 |
| 116 | VPS25 | TCCCTGCTAGAAGAACTTGA | 114 |
| 117 | MRPL10 | GCTGGCTACGAGTCCGGAAC | 115 |
| 118 | U2AF2 | CCGCCTCTACCAGAAGTCCC | 116 |
| 119 | DNM2 | GAGGCCTAGTCGAGCAGGGA | 117 |
| 120 | FBXO17 | TCGCTAGGACAGACGGATCC | 118 |
| 121 | CLASRP | TCTGCCTAATGTCGGTAATG | 119 |
| 122 | RPS16 | GTCAGCTACCAGCAGGGTCC | 120 |
| 123 | MRPL4 | GTGATTCTAACAGCGGAGCC | 121 |
| 124 | MRPL4 | TGTGGTCTAGTGTGACTTTG | 122 |
| 125 | RPS19 | TTGTTCTAATGCTTCTTGTT | 123 |
| 126 | RPL18A | TGCACCTAGAAGAAGGTGTT | 124 |
| 127 | ELL | GCGGCTAGGGCCAAGCCTGC | 125 |
| 128 | SNRPD2 | CGGCCCCTACTTGCCGGCGA | 126 |
| 129 | DOHH | GGGGCCCTAGGAGGGGGCCC | 127 |
| 130 | UBE2M | GCCAACCCTATTTCAGGCAG | 128 |
| 131 | ZC3H4 | GGACACTACTGGCAAAAGGG | 129 |
| 132 | SAE1 | ATGGACTAGTGTCTCGGCTT | 130 |
| 133 | LENG8 | GGTCTCTATGGTGGGAGCAC | 131 |
| 134 | EEF2 | GGCCGCCTACAATTTGTCCA | 132 |
| 135 | UBL5 | TTCTCATCTATTGATAATAA | 133 |
| 136 | RAE1 | AGCCACTACTTCTTATTCCT | 134 |
| 137 | TTI1 | AGGCTCTAAGCACTGCCAGG | 135 |
| 138 | ZNF335 | AGGTTCTAGGAGAAGATGGA | 136 |
| 139 | NFS1 | CTTCTAGTGTTGGGTCCACT | 137 |
| 140 | SON | ATTTGCTACCACCAAAATCT | 138 |
| 141 | SF3A1 | TCTTGTCTACTTCTTCCTCC | 139 |
| 142 | PPIL2 | CTGCTGCTACCAGGAGCTGA | 140 |
| 143 | PPIL2 | ACCTCTAGTGGTCATCAGGC | 141 |
| 144 | EP300 | TGTCTCTAGTGTATGTCTAG | 142 |
| 145 | RANGAP1 | TGAGTCTAGACCTTGTACAG | 143 |
| 146 | POLR3H | GGGCTAGTTGCTGGTCCACC | 144 |
| 147 | ADSL | CAACTCTACAGACATAATTC | 145 |
| 148 | SMC1A | ATACTGCTACTGCTCATTGG | 146 |
| 149 | PGK1 | AAGTACTAAATATTGCTGAG | 147 |
| 150 | RBMX | TTATCTACTGTGAATCAATC | 148 |
| 151 | RBMX | TTGTTTCTAGTATCTGCTTC | 149 |
| 152 | SKI | GGAATCTACGGCTCCAGCTC | 150 |

### Example 2

1. Synthesis of gRNA Array

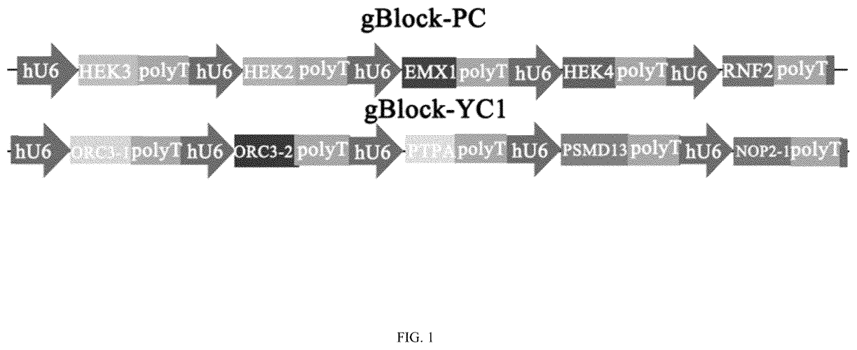

AgBlock (i.e., gRNA array) containing 5 sgRNA expression cassettes was designed, denoted as gBlock-YC1, and synthesized by a biotech corporation. gBlock-YC1 carried sgRNA targeting 5 gene loci (ORC3-1, ORC3-2, PTPA, PMSD13, or NOP2-1). Each expression cassette comprised hU6, an sgRNA and a polyT in the 5′ to 3′ direction. The sequences of sgRNAs for the 5 gene loci are shown in Table 1. Meanwhile, 5 previously reported sgRNAs (gBlock PC) were used as the positive controls (Thuronyi, B. W. et al., Continuous evolution of base editors with expanded target compatibility and improved activity, Nat Biotechnol, 37, 1070-1079 (2019)). The gBlock-PC carried sgRNAs of 5 endogenous loci (HEK2, HEK3, HEK4, EMX1, and RNF2). The backbone plasmid for gBlock-YC1 and gBlock-PC was puc57. The structures of gBlock-YC1 and gBlock PC are shown in FIG. 1.

### 2. Transfection of HEK293T Cells

HEK293T cells were transiently co-transfected with gBlock-YC1 or gBlock PC and a base editor plasmid (evoAPOBEC1-BE4max-NG). The transfection was performed using Lipofectamine 3000 (Thermo Fisher Scientific, Cat #L3000015) except for the following modifications: cells were seeded into a 48-well plate at 5×104 cells per well and incubated for 24 h in 250 μL of cell culture medium. For each gBlock plasmid and the base editor plasmid, the transfection was performed with 1 μg of DNA (750 ng of base editor plasmid, 250 ng of each gBlock plasmid) and 2 μL of Lipofectamine 3000 per well.

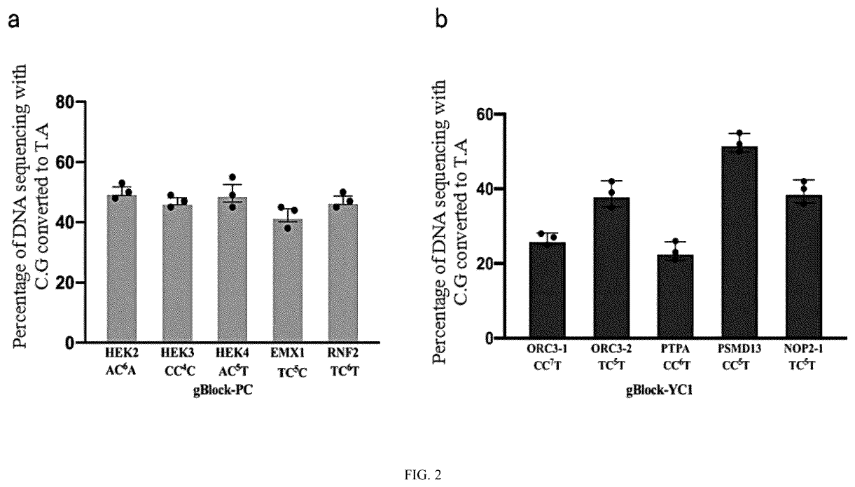

Sanger sequencing and EditR analysis of the targeted loci gave the frequency (%) of C-to-T conversion, as shown in FIG. 2. Editing efficiencies of the loci targeted by gBlock-PC and gBlock-YC1 were 40-50% and 20-50%, respectively, indicating that gBlock-YC1 can maintain high base editing efficiency.

### Example 3

1. Construction of Cell Lines having a Stable Doxycycline-Inducible CBE

Two HEK293T cell lines stably expressing doxycycline-inducible PB-FNLS-BE3-NG1 and PB-evoAPOBEC1-BE4max-NG were constructed by using PB transposon technique: HEK293T cells were seeded on a 6-well plate at 5×105 cells per well, incubated for 24 h, and transfected with 1 μg of super transposase plasmid (SBI System Biosciences, Cat #PB210PA-1) and 4 μg of piggy Bac targeted base editor plasmid according to the instructions of Lipofectamine 3000. After 48 h, the cells were screened with puromycin (2 μg/mL). The polyclonal pool was cultured for 7-10 days after screening, or the clonal cell lines were cultured for 5-7 days after screening. The cells were sorted into single cells on a 96-well plate by flow cytometry. Puromycin was added periodically during the long-term culture.

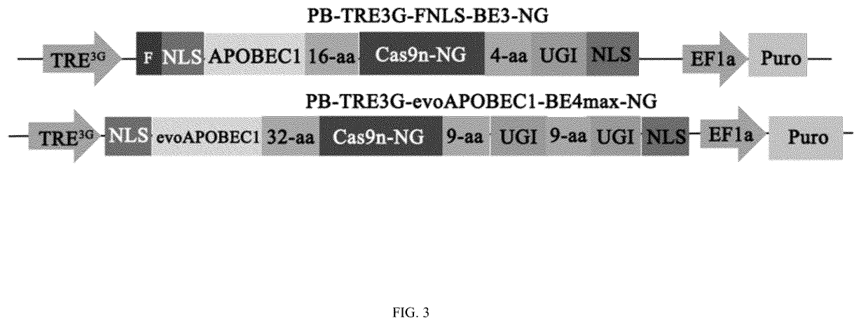

The structure of doxycycline-inducible cytidine deaminase piggy Bac is shown in FIG. 3.

2. Transfection of Cell Lines having a Stable Doxycycline-Inducible CBE

Two cell lines having a stable doxycycline-inducible CBE were transiently transfected with gBlock-PC or gBlock-YC1, respectively: The cells were seeded on a 48-well poly (d-lysine) plate (Corning, Cat #354413) at 1× 10⁵ cells per well, incubated in 300 μL of culture medium containing doxycycline (2 μg/mL) for 24 h, and transfected with a system of 1 μg of gBlock-PC or gBlock-YC1 and 2 μL of Lipofectamine 3000 per well. After the transfection, doxycycline was added, and the cells were incubated for 5 days and collected for genomic DNA editing analysis.

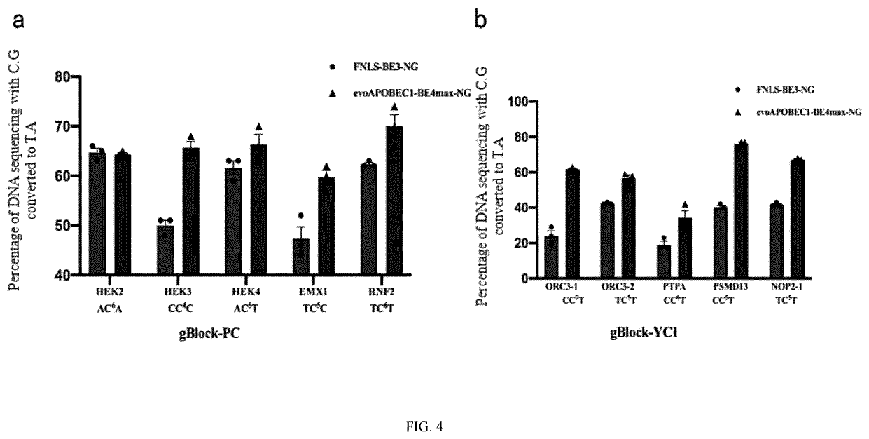

Sanger sequencing and EditR analysis of the targeted loci gave the frequency (%) of C-to-T conversion, as shown in FIG. 4. The editing efficiency of sgRNAs in gBlock-PC was about 60-70% in the cell line stably expressing evoAPOBEC1-BE4max-NG, which was slightly higher than 45-65% in the cell line stably expressing FNLS-BE3-NG. The editing efficiency of sgRNAs in gBlock-YC1 was about 30-75% in the cell line stably expressing evoAPOBEC1-BE4max-NG, which was significantly higher than 20-40% in the cell line stably expressing FNLS-BE3-NG. The cell line stably expressing evoAPOBEC1-BE4max-NG has higher base editing efficiency.

To provide higher base editing efficiency, a preferred embodiment of the present invention employs a cell line stably expressing evoAPOBEC1-BE4max-NG for the transfection of gBlock.

### Example 4

1. Sorting of Monoclones from Cell Line Stably Expressing evoAPOBEC1-BE4max-NG

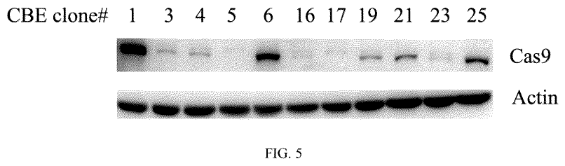

Monoclones were isolated from the cell line stably expressing evoAPOBEC1-BE4max-NG by flow cytometry, resulting in clones1, 3, 4, 5, 6, 16, 17, 19, 21, 23, and 25, which were then cultured. After 5 days of doxycycline induction, Western blotting was performed in triplicate, with the expression levels of the cytosine base editor in each clone shown in FIG. 5. The immunoblot images in FIG. 5 are representative of the three replicates.

### 2. Transfection of Monoclones

gBlock-YC1 was transiently transfected into the resulting monoclone in quadruplicate. The monoclonal cells were seeded on a 48-well poly (d-lysine) plate (Corning, Cat #354413) at 1×10⁵ cells per well, incubated in 300 μL of culture medium containing doxycycline (2 μg/mL) for 24 h, and transfected with a system of 1 μg of gBlock-YC1 and 2 μL of Lipofectamine 3000 per well. After the transfection, doxycycline was added, and the cells were incubated for 5 days and collected for genomic DNA editing analysis.

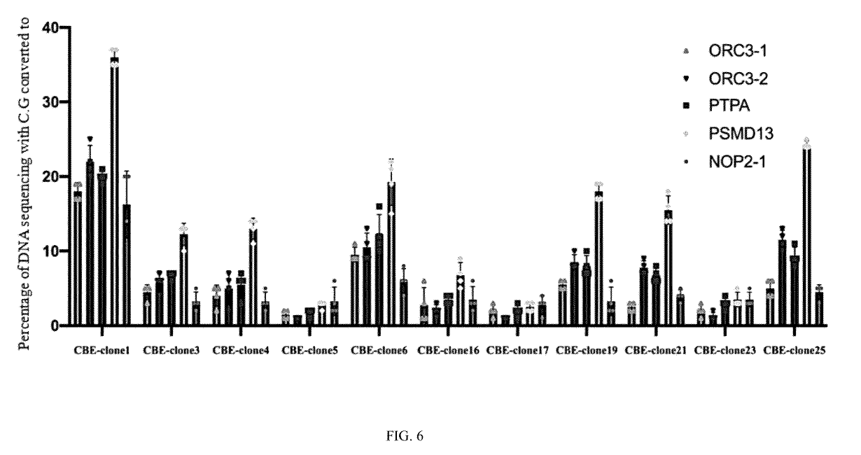

Sanger sequencing and EditR analysis of the targeted loci gave the frequency (%) of C.G-to-T.A conversion, as shown in FIG. 6. The editing efficiency of 5 gene loci in clone 1 was the highest among the 11 clones.

### Example 5

10-gBlocks pool: the target gene loci are Nos. 1-52 in Table 1, and the sgRNA sequences are shown in Table 1.

20-gBlocks pool: the target gene loci are Nos. 1-102 in Table 1, and the sgRNA sequences are shown in Table 1.

30-gBlocks pool: the target gene loci are Nos. 1-152 in Table 1, and the sgRNA sequences are shown in Table 1.

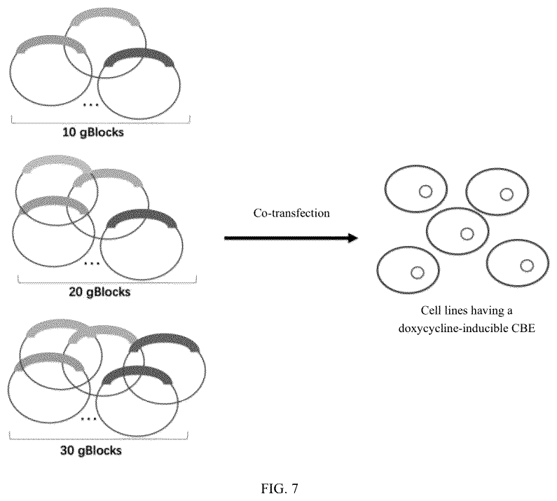

The 10-, 20-, or 30-gBlocks pool was co-transfected into clone 1 of the cell line stably expressing evoAPOBEC1-BE4max-NG selected in Example 4, as shown in FIG. 7. Specifically, the 10-, 20-, or 30-gBlocks pool was delivered into the stable cell lines in doxycycline-containing medium or doxycycline-free medium.

The cells were seeded on a 48-well poly (d-lysine) plate (Corning, Cat #354413) at 1×10⁵ cells per well, and incubated in 300 μL of culture medium containing doxycycline (2 μg/mL), 20 mM p53 inhibitor (Stem Cell Technologies, Cat #72062) and 20 ng/ml human recombinant bFGF (Stem Cell Technologies, Cat #78003) for 24 h. For the 10-gBlocks pool, the transfection was performed using a system of 200 ng of plasmid per gBlocks and 3 μL of Lipofectamine 3000 per well, and 20 ng of green fluorescent protein was used as the transfection control; for the 20-gBlocks pool, the transfection was performed using a system of 150 ng of plasmid per gBlocks and 3 μL of Lipofectamine 3000 per well, and 20 ng of green fluorescent protein was used as the transfection control: for the 30-gBlocks pool, the transfection was performed using a system of 100 ng of plasmid per gBlocks and 3 μL of Lipofectamine 3000 per well, and 20 ng of green fluorescent protein was used as the transfection control. After the transfection, doxycycline was added, and the cells were incubated for 5 days and collected for genomic DNA editing analysis.

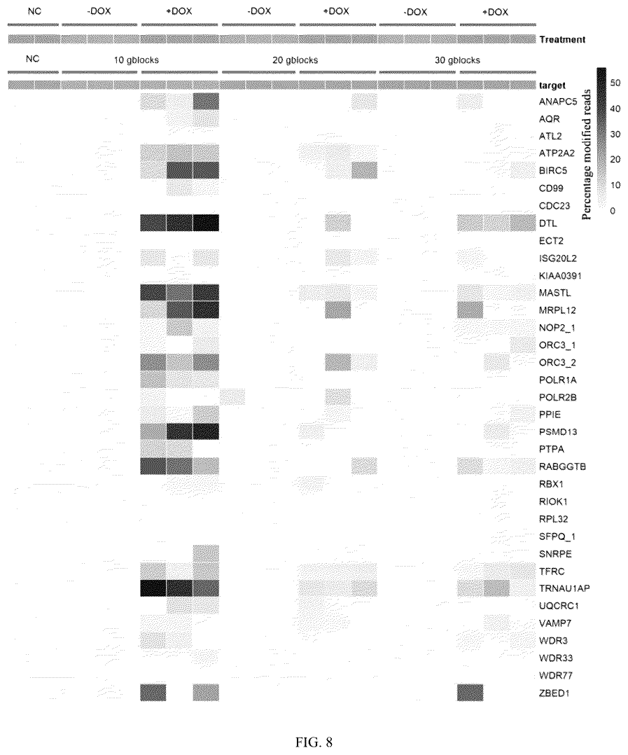

A heatmap of “C” mutation frequencies in targeted loci was obtained by whole exome sequencing (WES), as shown in FIG. 8. The editing efficiency was the best in most of the 52 gene loci when delivering 10 gBlocks compared to those of 20 gBlocks and 30 gBlocks.

To provide higher base editing efficiency, a preferred embodiment of the present invention employs the 10 gBlocks in one delivery.

### Example 6

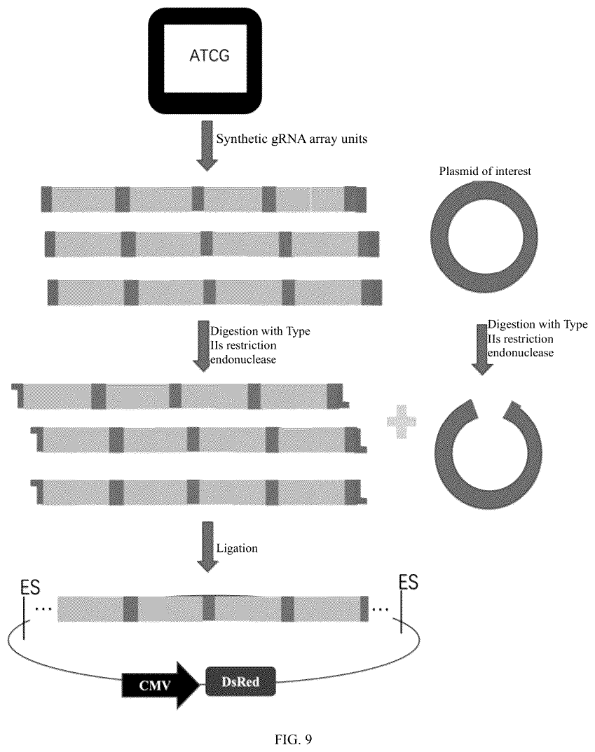

The 10-gBlocks pool was assembled into DsRed-containing expression vectors by Golden gate assembly, as in FIG. 9.

The sgRNA sequences targeting the gene loci were designed by software, connected in series and sent to a contractor to synthesize multiple gRNA array units (gBlocks). Each gBlock array contained 5 sgRNA expression cassettes connected in series. Each gBlock fragment contained 5 sgRNA expression cassettes, and was directly synthesized into the PUC57 cloning plasmid after digestion sites of type IIS restriction endonuclease BbsI were added at the two ends. Two oligonucleotide chains Spel-HF with BbsI digestion sites were annealed and cloned into a target vector expressing a CMV promoter-driven fluorescent protein (DsRed). The 10-gBlocks pool and the plasmid of interest were separately digested with BbsI-HF, and extracted with a gel extraction kit (Zymo Research, Cat #11-301C). The gBlocks fragments were treated with T4 DNA ligase (NEB, Cat #M0202S) overnight at 16° C. and ligated to the plasmid. After the completion of the ligation reaction, 2 μL of the reaction mixture was transformed into an E. coli NEB Stable strain. The plasmid DNA was isolated from the suspension using the QIAprep spin purification kit (Cat #27104) according to the instructions.

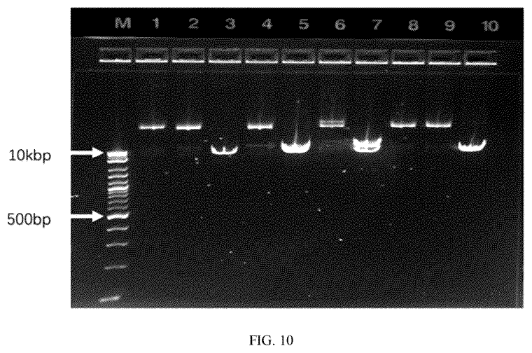

Whether the sgRNAs were successfully inserted into the final integrative plasmid was analyzed by agarose gel electrophoresis. Nine plasmids were selected for detection, and were all linearized by endonuclease spel. Since Spel sites are arranged at the two ends of the multiple sgRNAs insertion sites, when multiple sgRNAs were successfully inserted into the plasmids, two bands were seen in gel electrophoresis after the plasmids were digested by the Spel. One fragment was approximately 4479 bp, and the other fragment was approximately 22140 bp. Two of the nine plasmids tested had the correct insert size, indicating that the sgRNAs were successfully inserted. The results are shown in FIG. 10.

The insertion of multiple sgRNAs was verified by Sanger sequencing. The sequencing results demonstrate that the constructed integrative plasmid contained 43 sgRNAs. The plasmid was denoted as 43-all-in-one, and the sequence of the plasmid 43-all-in-one is set forth in SEQ ID NO. 151.

### Example 7

Ten gRNA arrays were delivered to the cells stably expressing doxycycline-inducible evoAPOBEC1-BE4max-NG using the following 3 methods: The cells were seeded on a 48-well poly (d-lysine) plate (Corning, Cat #354413) at 1×10⁵ cells per well, incubated in 300 μL of polytetracycline (2 μg/mL) for 24 h, and transfected with a system of 21 μg of the plasmid and 3 μL of Lipofectamine 3000 per well. After the transfection, polytetracycline was added, and the cells were incubated for 5 days and collected for genomic DNA editing analysis.

Method 1: The 10-gBlocks pool (200 ng each), a plasmid eGFP L202 Reporter containing mCherry-inactivated eGFP reporter (Addgene, #119129; 30 ng), and 3 μL of Lipofectamine 3000.

Method 2: The 10-gBlocks pool (200 ng each), a plasmid eGFP L202 Reporter containing mCherry-inactivated eGFP reporter (Addgene, #119129:30 ng), eGFP L202 gRNA (Addgene, #119132:10 ng), and 3 μL of Lipofectamine 3000.

Method 3: 2 μg of 43-all-in-one plasmid and 3 μL of Lipofectamine 3000.

10-gBlocks pool: the target gene loci are Nos. 1-52 in Table 1, and the sgRNA sequences are shown in Table 1.

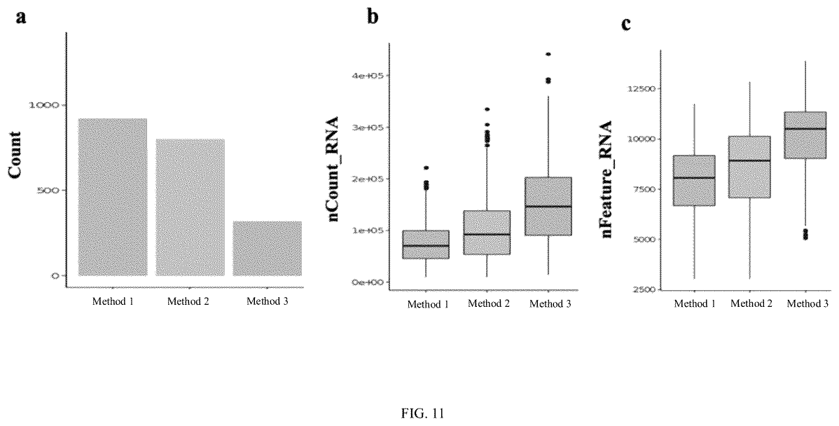

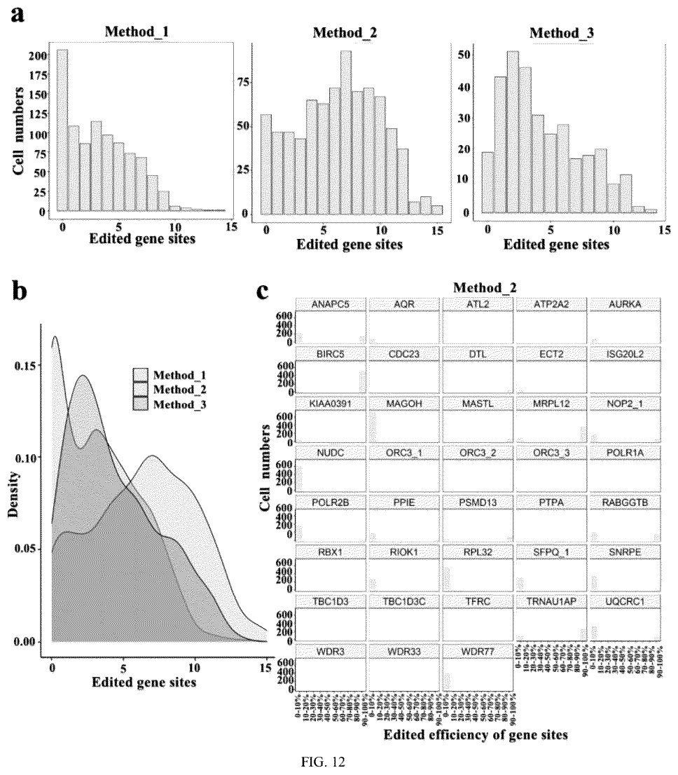

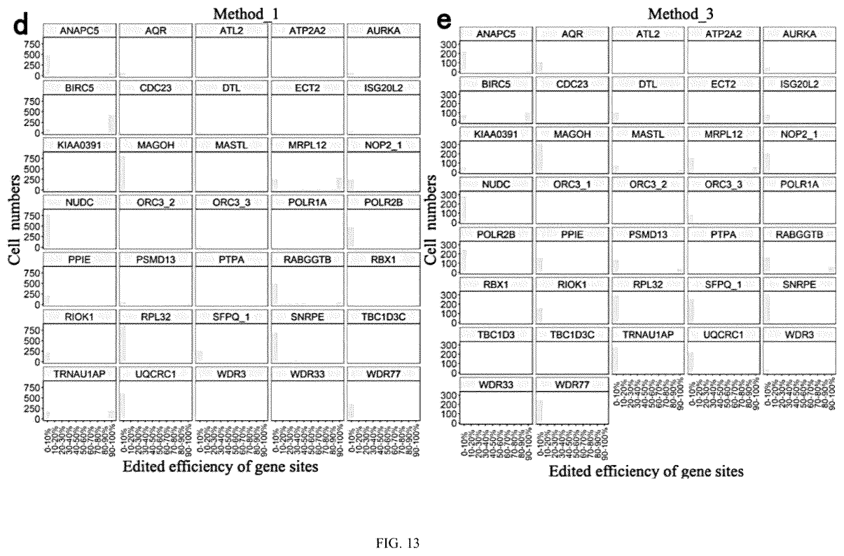

Approximately 1000 individual cells were isolated by each method, and the basic quality attributes of single cell RNA sequencing with 3 different delivery methods are shown in FIG. 11. Using the CRISPResso2 software, 38 of the 47 gene loci in HEK293T cells were matched, and a decrease in the number of cells with an increase in the number of editing sites within a single cell was observed in the three methods. Method 2 showed the greatest number of cells edited by multiple gene loci simultaneously. The population density graph of the cells was plotted, and the editing efficiency of each target was analyzed, suggesting that the editing events at the target loci were in bimodal distribution (FIG. 12-13).

At the same time, the editing efficiency of all targeted loci in each cell and the overall editing efficiency of all targeted loci under each delivery method were analyzed, as in FIG. 14. The results show that method 2 is the most efficient one among the three delivery methods.

To provide higher base editing efficiency, a preferred embodiment of the present invention employs method 2 for the delivery of gRNA arrays.

### Example 8

28/96 and 24/96 monoclones were isolated from the cell populations transfected by method 2 and method 3, respectively, in Example 7 and cultured.

For the clones of method 2, 10 easily editable loci (PSMD13, ANAPC5, BIRC5, WDR3, MASTL, RBX1, PPIE, RABGGTB, SNRPE, and UQCRC1 in Table 1) were selected, amplified by PCR, and sequenced by Sanger sequencing and EditR analysis. It was found that 4 clones were not transferred with any of the gBlocks and 24 clones were transferred with 1-10 gBlocks, among which clone 19 was transferred with all of the 10 gBlocks.

For the clones of method 3, 3 easily editable loci (PSMD13, ANAPC5, and BIRC5 in Table 1) were selected for screening. It was found that in 13 clones, none of the 3 loci was edited, and in 11 clones, several loci were not edited, among which clones 11, 20, 21, and 24 had 3 edited loci.

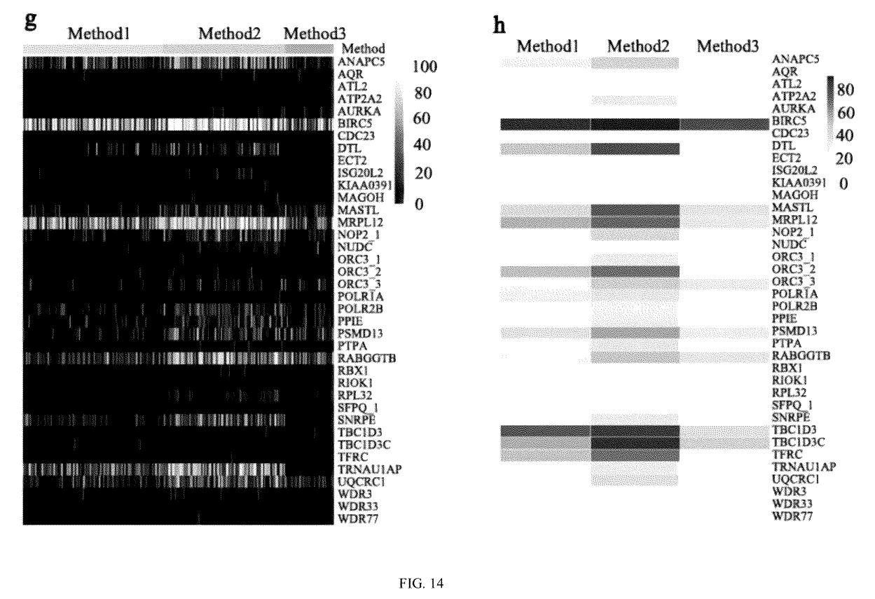

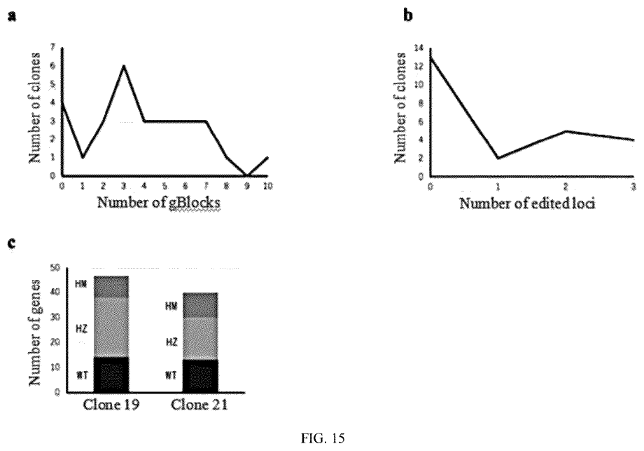

The target loci in two highly modified clones: clone 19 (from method 2) and clone 21 (from method 3) were subjected to Sanger sequencing. The results show that in clone 19, TAG to TAA conversion was found at 33/47 genomic loci with 9 loci being homozygous loci, and 14/47 loci were unedited; in clone 21, TAG to TAA conversion was found in 27/40 loci with 10 loci being homozygous loci, and 13/40 loci were unedited (FIG. 15).

To determine whether the editing efficiency could be increased with runs of transfection, gBlocks were transfected into highly modified clone 19 (from method 1) using method 1, and clones 19-1, 19-16, and 19-21 were selected from 22/96 clones with higher edits in the selected loci compared to the original clone 19 (Sanger/EditR).

To provide higher base editing efficiency, a preferred embodiment of the present invention employs method 2 in Example 7 to deliver ten gRNA arrays into the cells, then isolates monoclones from the transfected cell population and cultures the monoclones, and again employs method 2 in Example 7 to deliver ten gRNA arrays into isolated highly modified monoclones.

### Example 9

To completely evaluate the targeted editing and off-target efficiency of TAG to TAA transformation in the CBE whole genome, 30-fold whole genome sequencing (WGS) was performed on the highly modified clones in Example 8 (19, 21, 19-1, 19-16, and 19-21) and a negative control (HEK293T cells).

In the targeted editing, 39/47 gene loci were matched in the highly modified clones, 28 of which showed higher edits, and clones 19-1, 19-16, and 19-21 had improved editing ability at the selected loci compared to clone 19, which is consistent with the results of Sanger sequencing in Example 8.

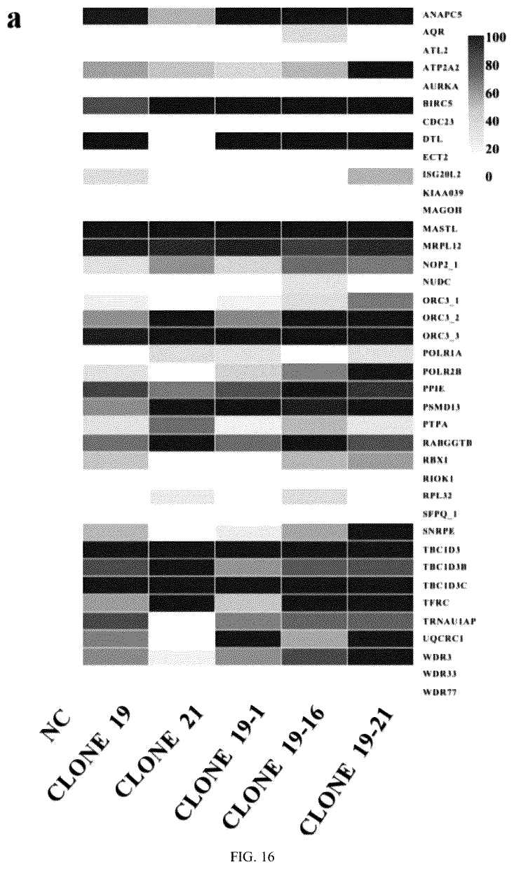

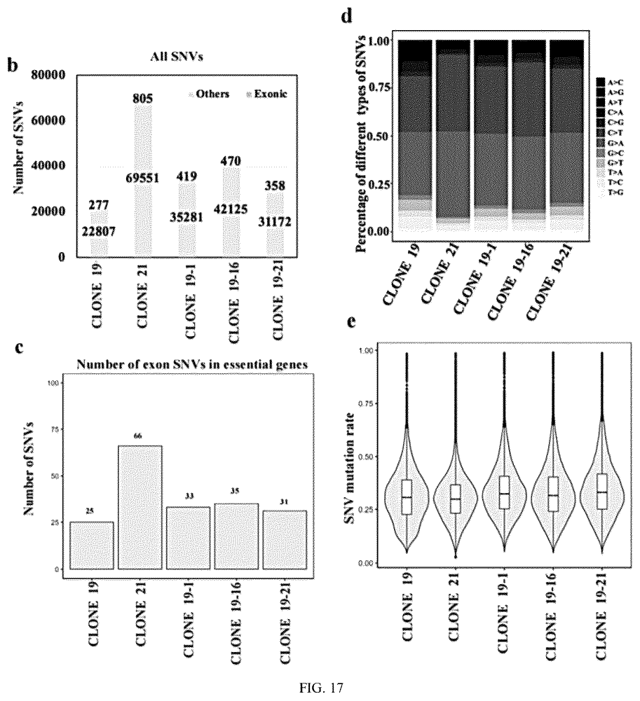

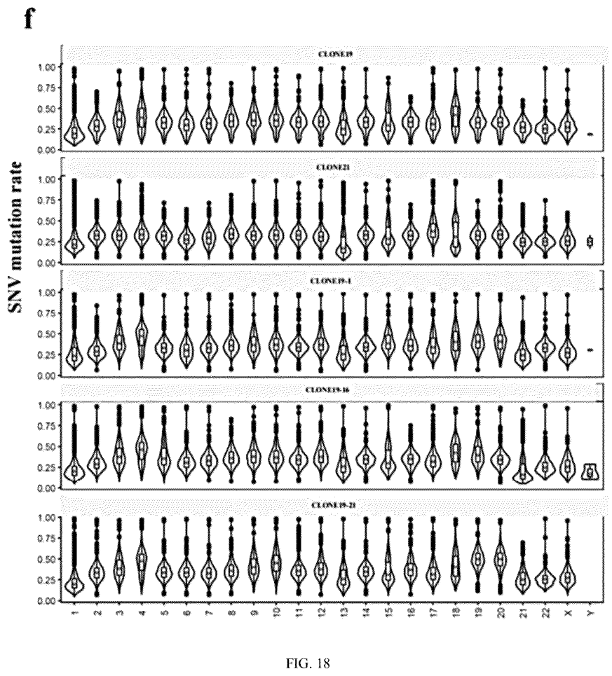

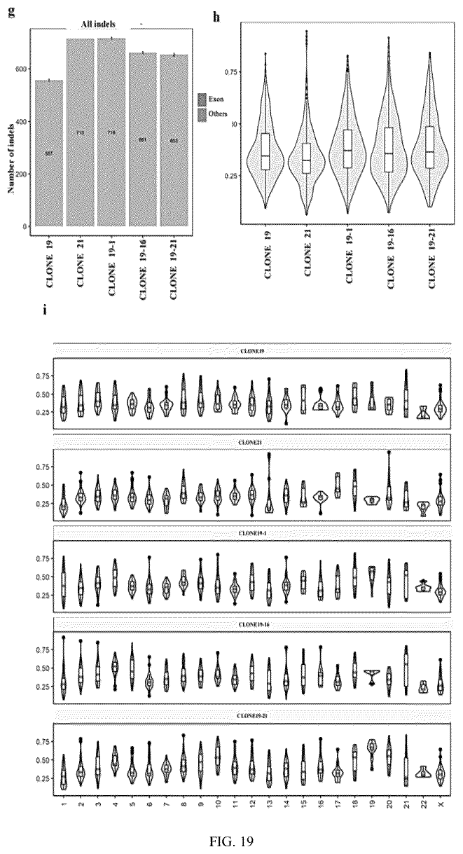

To explore the off-target events, highly modified clones (19, 21, 19-1, 19-16, and 19-21) were analyzed for single nucleotide variations (SNVs) and insertions/deletions (indels). The SNVs in clone 19, clone 21, clone 19-1, clone 19-16, and clone 19-21 were 23084, 70356, 35700, 42595, and 31530, respectively, after subtraction of the target loci as compared to the control group. Further analysis showed that 277, 805, 419, 470, and 358 SNVs were respectively positioned in exons, and only 33, 77, 42, 46, and 40 SNVs were positioned on the exons of essential genes. The SNVs were classified into different mutation types, and the C-to-T (G-to-A) conversion was found to be the most common edit (FIG. 16-19). The SNV mutation rates were low, but were seen in all clones and distributed on all chromosomes. In addition to the SNVs, the number of indels detected in these clones was 558, 715, 717, 662, and 655, respectively, of which a small portion was located in the exons and none were found in the exon of essential genes. The indel ratios were also low for all clones and chromosomes (FIG. 20-21).

### Example 10

Ten gBlocks were delivered by method 2 into clone 1 sorted from the cells stably expressing evoAPOBEC1-BE4max-NG in Example 3: The cells were seeded on a 48-well poly (d-lysine) plate (Corning, Cat #354413) at 1×10⁵ cells per well, incubated in 300 μL of polytetracycline (2 μg/mL) for 24 h, and transfected with a system of 21 μg of the plasmid and 3 μL of Lipofectamine 3000 per well. After the transfection, polytetracycline was added, and the cells were incubated for 5 days and collected.

Method 2: The 10-gBlocks pool (200 ng each), a plasmid eGFP L202 Reporter containing mCherry-inactivated eGFP reporter (Addgene, #119129; 30 ng), eGFP L202 gRNA (Addgene, #119132; 10 ng), and 3 μL of Lipofectamine 3000.

A more preferred embodiment further comprises isolating monoclones from the transfected cell population and culturing, selecting monoclones with high editing efficiency, and delivering the ten gRNA arrays into the isolated highly modified monoclones again using method 2. After the transfection, polytetracycline was added, and the cells were incubated for 5 days and collected. This procedure can be repeated for a plurality of times as desired.

It is obvious that the above examples are merely illustrative for a clear explanation and are not intended to limit the implementations. Various changes and modifications can be made by those of ordinary skill in the art on the basis of the above description. It is unnecessary and impossible to exhaustively list all the implementations herein. Obvious changes or modifications derived therefrom still fall within the protection scope of the present invention.

What is claimed is:

1. A gRNA array, comprising 5 sgRNA expression cassettes connected in series, characterized in that each sgRNA expression cassette comprises a promoter, an sgRNA and a polyT in the 5′ to 3′ direction; the sgRNA in the sgRNA expression cassette is selected from the sequence set forth in one of SEQ ID NOs. 1-150, and the sgRNAs of the gRNA array are different from each other.

2. A gRNA array pool, comprising 2-10 gRNA arrays, characterized in that each gRNA array comprises 5 sgRNA expression cassettes connected in series, characterized in that each sgRNA expression cassette comprises a promoter, an sgRNA and a polyT in the 5′ to 3′ direction; the sgRNA in the sgRNA expression cassette is selected from the sequence set forth in one of SEQ ID NOs. 1-150, and the sgRNAs of the gRNA array pool are different from each other.

3. An expression vector, characterized in that the expression vector has a nucleotide sequence set forth in SEQ ID NO. 151.

4. A base editing system, comprising the gRNA array pool according to claim 2 or a transcript thereof;
further comprising a base editor, characterized in that the base editor is selected from an adenine base editor or a cytosine base editor.

5. A kit for multiplex base editing, comprising the base editing system according to claim 4, a plasmid containing an mCherry-inactivated eGFP reporter and an sgRNA plasmid for editing and activating eGFP.

6. A method for high-throughput TAG to TAA conversion on the genome, comprising:
transfecting a cell with a gRNA array by the following method to achieve TAG to TAA conversion;
co-transfecting the gRNA array pool according to claim 2 or a transcript thereof, a plasmid containing an mCherry-inactivated eGFP reporter, an sgRNA plasmid for editing and activating eGFP, and a base editor into the cell; or
co-transfecting the expression vector according to claim 3 or a transcript thereof and a base editor into the cell; or
co-transfecting the gRNA array pool according to claim 2 or a transcript thereof, a plasmid containing an mCherry-inactivated eGFP reporter, and an sgRNA plasmid for editing and activating eGFP into a cell having a stable inducible base editor; or
transfecting the expression vector according to claim 3 or a transcript thereof into a cell having a stable inducible base editor.

7. The method according to claim 6, further comprising: isolating monoclones from the transfected cells and culturing, performing Sanger sequencing and EditR analysis, selecting monoclones with high editing efficiency, and transfecting with a gRNA array by the method I or II.

8. The method according to claim 6, characterized in that the cell is a mammalian cell.

9. The method according to claim 6, characterized in that, as per 1×10⁵ mammalian cells, the transfection amount of the gRNA array is 200 ng, the transfection amount of the plasmid containing an mCherry-inactivated eGFP reporter is 30 ng, and the transfection amount of the sgRNA plasmid for editing and activating eGFP is 10 ng;
as per 1×10⁵ mammalian cells, the transfection amount of the expression vector according to claim 3 is 2 μg.

10. The method according to claim 6, characterized in that the cell having a stable inducible base editor is selected from a cell monoclone having a stable inducible base editor with high editing efficiency.

11. The method according to claim 10, characterized in that the method for screening the cell monoclone having a stable inducible base editor with high editing efficiency comprises: selecting cell monoclones having a stable inducible base editor denoted as original monoclones; and transfecting one gRNA array into the selected original monoclones, and selecting transfected monoclones with high editing efficiency, characterized in that the original monoclones corresponding to the transfected monoclones with high editing efficiency are the cell monoclones having a stable inducible base editor with high editing efficiency.

12. The method according to claim 6, characterized in that the inducible base editor is a doxycycline-inducible base editor.

13. The method according to claim 6, characterized in that the inducible base editor is a doxycycline-inducible cytosine base editor.

14. The method according to claim 6, characterized in that the cell having a stable inducible base editor is selected from a mammalian cell stably expressing PB-FNLS-BE3-NG1 or PB-evoAPOBEC1-BE4max-NG.
